(19) United States
(12) Patent Application Publication
(10) Pub. No.: US 2017/0143947 A1
(43) Pub. Date: May 25, 2017

(54) DEVICES, SYSTEMS AND METHODS FOR TREATING ULCERATIVE COLITIS AND OTHER INFLAMMATORY BOWEL DISEASES

(71) Applicant: COVIDIEN LP, Mansfield, MA (US)

(72) Inventors: REBECCA J. COULSON, LYONS, CO (US); DARION R. PETERSON, LONGMONT, CO (US); JAMES D. ALLEN, IV, BROOMFIELD, CO (US); ANTHONY B. ROSS, BOULDER, CO (US); WILLIAM H. NAU, JR., LONGMONT, CO (US)

(21) Appl. No.: 15/340,408

(22) Filed: Nov. 1, 2016

Related U.S. Application Data
(60) Provisional application No. 62/258,005, filed on Nov. 20, 2015.

(51) Int. Cl. A61M 31/00; A61N 5/06; A61B 18/18; A61F 7/00; A61B 18/14

(52) U.S. Cl. CPC A61M 31/00; A61F 7/007; A61B 18/1485; A61B 18/1815; A61N 5/0603; A61N 5/062; A61N 5/0625; A61F 2007/0071; A61F 2007/0018; A61B 2018/00494

(57) ABSTRACT

An apparatus for treating inflammatory bowel diseases is provided. The apparatus includes a handle, a shaft extending distally from the handle, and an end effector disposed on a distal end of the shaft. The shaft is configured to be advanced within the colon of a patient. The end effector includes a radiating portion and a nozzle disposed thereon. The radiating portion is configured to emit energy therefrom and the nozzle is configured to emit a fluid therefrom. A method of treating inflammatory bowel diseases is also provided.

### CROSS REFERENCE TO RELATED APPLICATION

The present application claims the benefit of and priority to U.S. Provisional Application Ser. No. 62/258,005, filed on Nov. 20, 2015, the entire contents of which are incorporated herein by reference.

### BACKGROUND

Technical Field

The present disclosure relates to treatment of Inflammatory Bowel Diseases (IBDs) and, more particularly, to surgical devices, systems, and methods for treating ulcerative colitis and other IBDs, e.g., Crohn's Disease.

Background of Related Art

Ulcerative colitis, an IBD, is a disease of the colon in which inflammation and ulcers, or sores, form on the interior wall of the colon. Ulcerative colitis manifests itself, usually intermittently and at varying degrees of severity, in symptoms such as stomach pain, diarrhea, and/or bloody stool. Typically, anti-inflammatory medication(s) are prescribed for patients suffering from ulcerative colitis. In extreme cases, chronic cases, or cases in which medication(s) fails to adequately treat the patient's symptoms, surgery to remove all or part of the diseased portions of the rectum and/or colon may be performed.

Although medication(s) and surgical removal of diseased portions of the rectum and/or colon are effective in certain instances, there is a need for surgical devices, systems, and methods to more effectively and/or efficiently treat ulcerative colitis and other IBDs, e.g., Crohn's Disease, while minimizing side effects and damage to healthy tissue.

### SUMMARY

As used herein, the term “distal” refers to the portion that is being described which is further from a user, while the term “proximal” refers to the portion that is being described which is closer to a user. Further, to the extent consistent, any of the aspects described herein may be used in conjunction with any or all of the other aspects described herein.

An apparatus for treating inflammatory bowel diseases provided in accordance with the present disclosure includes a handle, a shaft extending distally from the handle, and an end effector disposed at a distal end of the shaft. The shaft is configured to be advanced within a patient's colon. The end effector includes a radiating portion configured to emit energy and a nozzle configured to emit a fluid.

In aspects, the radiating portion and the nozzle are disposed in a concentric orientation with the nozzle disposed within the radiating portion.

In some aspects, the nozzle is disposed distal of the radiating portion.

In certain aspects, the end effector includes an expanding portion. The expanding portion is configured to engage an inner wall of a patient's colon.

In aspects, the radiating portion is disposed within the expanding portion.

In certain aspects, the expanding portion is configured to diffuse light.

In some aspects, the handle includes a first switch disposed thereon. The first switch is operably coupled to the radiating portion. Activating the first switch causes the radiating portion to emit energy therefrom.

In aspects, the handle includes a second switch disposed thereon. The second switch is operably coupled to the nozzle. Activating the second switch causes the nozzle to emit fluid therefrom.

In some aspects, the radiating portion is configured to emit radio frequency, microwave, or light energy.

In certain aspects, the fluid is a fluid containing nano-particles or a photosensitizer.

According to another aspect of the present disclosure, an apparatus for treating inflammatory bowel diseases includes a body, a conductor disposed on the body, and at least one terminal disposed on an end of the conductor. The body is configured to be advanced within a patient's colon, the conductor is capable of transmitting energy therethrough, and the at least one terminal is configured to selectively engage an energy source.

In aspects, the body defines a generally flat configuration including an adhesive disposed on a surface thereof. The adhesive is configured to adhere the body to an inner wall of a patient's colon.

In certain aspects, the body defines a cylindrical configuration. The body is biased towards an expanded configuration for engaging an inner wall of a patient's colon and is configured to be compressed by an external stimuli.

In some aspects, the conductor is configured to be resistively heated via the energy source to heat tissue adjacent thereto or to conduct electrical energy from the energy source through tissue adjacent thereto.

A method for treating inflammatory bowel diseases provided in accordance with the present disclosure includes coating an inner wall of a patient's colon with a fluid and emitting energy towards the fluid to heat the fluid, thereby thermally treating tissue of a patient's colon in contact with the fluid.

In aspects, emitting energy towards the fluid includes expanding an expandable portion of an end effector of a surgical instrument such that the expanding portion engages an inner wall of a patient's colon.

In certain aspects, emitting energy includes diffusing light energy through the expanding portion to heat tissue in contact with the expanding portion.

In some aspects, coating an inner wall of a patient's colon includes ejecting the fluid from a nozzle disposed on a distal end of an end effector of a surgical instrument.

In aspects, the fluid includes nano-particles suspended therein to facilitate absorption of the energy. Emitting the energy toward the fluid includes exciting the nano-particles to heat up the fluid.

In certain aspects, the fluid is a photosensitizer. Emitting the energy towards the fluid includes emitting light energy to excite the photosensitizer fluid.

### BRIEF DESCRIPTION OF THE DRAWINGS

Various aspects and features of the present disclosure described herein with reference to the drawings wherein:

FIG. 1 is a schematic illustration of a gastrointestinal system of a patient, showing the stomach, small intestine, large intestine, colon, and rectum;

FIG. 2A is a top view of a treatment system provided in accordance with the present disclosure;

FIG. 2B is a side view of the treatment system of FIG. 2A;

FIG. 2C is a schematic illustration of the treatment system of FIG. 2A positioned within the colon of the gastrointestinal system of FIG. 1;

FIG. 3A is a top view of another treatment system provided in accordance with the present disclosure;

FIG. 3B is a side view of the treatment system of FIG. 3A, shown in a collapsed state;

FIG. 3C is a side view of the treatment system of FIG. 3A, shown in an expanded state;

FIG. 3D is a schematic illustration of the treatment system of FIG. 3A disposed in the collapsed state and positioned within the colon of the gastrointestinal system of FIG. 1;

FIG. 3E is a schematic illustration of the treatment system of FIG. 3A disposed in the expanded state and positioned within the colon of the gastrointestinal system of FIG. 1;

FIG. 4A is a side view of yet another treatment system provided in accordance with the present disclosure;

FIG. 4B is a schematic illustration of the treatment system of FIG. 4A positioned within the colon of the gastrointestinal system of FIG. 1;

FIG. 5A is a side view of a treatment instrument provided in accordance with the present disclosure;

FIG. 5B is a schematic illustration of the treatment instrument of FIG. 5A positioned within the colon of the gastrointestinal system of FIG. 1;

FIG. 6A is a side view of another treatment instrument provided in accordance with the present disclosure;

FIG. 6B a schematic illustration of the treatment instrument of FIG. 6A positioned within of the colon of the gastrointestinal system of FIG. 1;

FIG. 7A is a side view of yet another treatment instrument provided in accordance with the present disclosure;

FIG. 7B is a schematic illustration of the treatment instrument of FIG. 7A positioned within the colon of the gastrointestinal system of FIG. 1;

FIG. 8A is a side view of still another treatment instrument provided in accordance with the present disclosure; and

FIG. 8B is a schematic illustration of the treatment instrument of FIG. 8A positioned within the gastrointestinal system of FIG. 1.

### DETAILED DESCRIPTION

As IBD's such as ulcerative colitis may only affect portions of the colon, it is desirable to focus treatment towards such diseased areas while limiting damage to surrounding healthy tissue and critical structures. Accordingly, the present disclosure provides various devices, systems, and methods configured to facilitate the focused or controlled treatment of diseased portions of the colon while limiting damage to surrounding portions of the colon and other surrounding tissue and critical structures. Although the various devices, systems, and methods provided herein are configured to treat any type of diseased bowel tissue, particular reference will be made to the colon “C.”

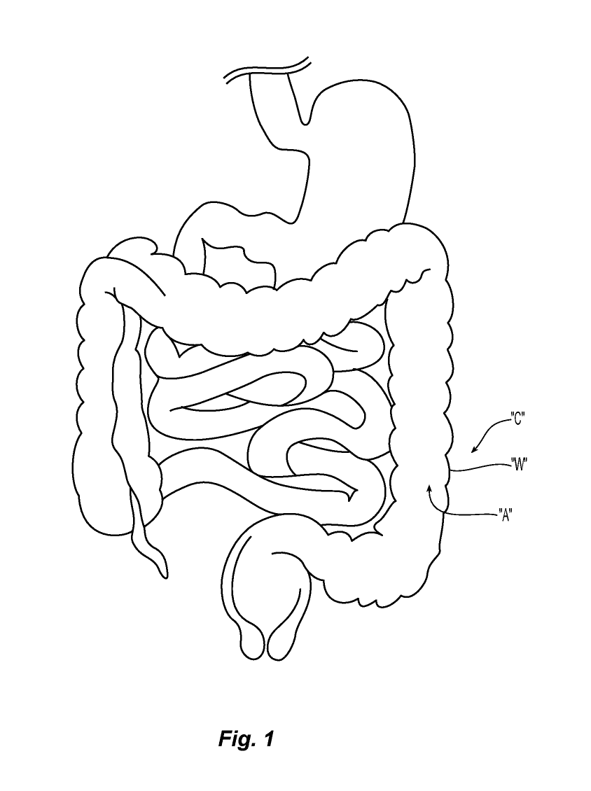

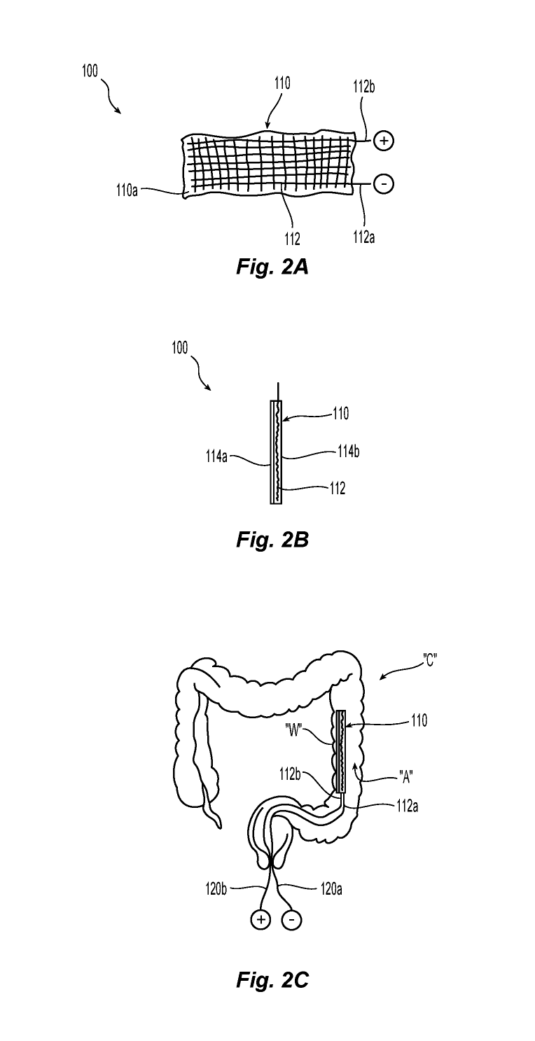

With reference to FIGS. 1-2C, a treatment system provided in accordance with the present disclosure and configured to treat diseased bowel tissue is shown generally identified as reference numeral 100. Treatment system 100 generally includes tape 110 having a conductor 112. Tape 110 is constructed of any suitable material and in some embodiments is formed from bio-absorbable material. Tape 110 is configured to be advanced within the colon “C” (FIG. 2C) of a patient. Although generally shown as including a rectangular configuration, it is contemplated that tape 110 may include any suitable configuration depending on the shape and/or size of the diseased area to be treated and/or the procedure being performed, such as circular, oval, square, or the like. Tape 110 includes a relatively thin cross section such that tape 110 can be easily maneuvered within the colon “C” and minimally protrudes within the annular area “A” (FIG. 2C) defined by the colon “C” when adhered to the inner wall “W” of the colon “C” as will be discussed in further detail below. The bottom surface of tape 100, e.g., the surface opposite top surface 110a, is configured to be selectively secured to an inner wall “W” (FIG. 2C) of the colon “C” using, for example, bio-absorbable adhesives or mechanical elements such as hooks or the like, which may be disposed on the bottom surface during manufacturing or may be applied thereto at any time prior to or during use.

Referring to FIG. 2A, conductor 112 is fixedly secured to or within top surface 110a of tape 110 such that conductor 112 is in thermal communication therewith. Conductor 112 is constructed of any suitable material and in some embodiments is formed from bio-absorbable material. Conductor 112 is configured to conduct electrical energy and includes a suitable impedance such that when electrical energy is conducted therethrough, tape 110 is evenly heated to a temperature capable of treating diseased tissue. Conductor 112 is arranged such that a first end 112a of conductor 112 and a second end 112b of conductor 112 are disposed on a proximal end of tape 110. Conductor 112 may be disposed on or within the top surface 110a of tape 110 in any suitable orientation capable of evenly distributing energy such that tape 110 is heated in an even manner, such as an undulating orientation, oval orientation, rectangular orientation, or the like, and may define any suitable configuration, e.g. serpentine, meshed, zig-zag, etc. Each end 112a, 112b of conductor 112 may be oriented in any suitable direction, such as opposing, normal, or the like. Each end 112a, 112b of conductor 112 extends past the perimeter of tape 110 and may terminate in an uncoated portion or connector (not shown). In some embodiments, each end 112a, 112b of conductor 112 may be terminated at an edge of tape 110 using any suitable method capable of maintaining the bio-absorbable capability of treatment system 100, such as a conductive pad, embedded tangs, terminals, or the like. In either configuration, the exposed contact portions, e.g., the portion extending past the perimeter of tape 110 or the edge of tape 110, enable leads of a suitable energy source (not shown) or resistive heater (not shown) to selectively engage conductor 112 to establish electrical communication with conductor 112, e.g., via an alligator clip, spade terminal, or the like.

With reference to FIG. 2B, tape 110 may be constructed in a composite or laminate configuration such that conductor 112 is embedded within tape 110. In such configurations, conductor 112 may be disposed within a pocket (not shown) formed within tape 110, which is then sealed over conductor 112 by a separate layer of bio-absorbable material to fully form tape 110 with conductor 110 is disposed between two portions 114a, 114b of tape 110. Alternatively, tape 110 may be constructed of two independent layers, which are laid over conductor 112, thereby sandwiching conductor 112 between the two portions 114a, 114b thereof.

In some embodiments, rather than functioning to be resistively heated for treating tissue adjacent thereto, conductor 112 may be configured to treat tissue by transmitting monopolar or bipolar electrosurgical energy to tissue adjacent thereto. In this manner, leads of a suitable electrosurgical generator (not shown) may be selectively engaged to ends 112a, 112b of conductor 112. In bipolar configurations, to inhibit shorting, conductor 112 may include two portions that are electrically-insulated from one another, one of the portions serving as the positive electrode and the other portion serving as the negative electrode.

Tape 110 may be constructed in either a non-porous or porous configuration, such as a bio-absorbable mesh, weave, or the like. As can be appreciated, tape 110 may be formed of a homogenous construction or a heterogeneous construction (i.e., a combination of metallic and non-metallic bio-absorbable materials).

Referring now to FIG. 2C, in use, once an area of interest is identified and the colon “C” has been prepared, suitable leads 120a, 120b are selectively engaged with the ends 112a, 112b of conductor 112. If monopolar or bipolar electrosurgical energy is being used, tape 110 may be soaked with a saline solution or other suitable solution before being inserted into the colon “C”. Soaking tape 110 with a saline solution increases the conductivity of tape 110 thereby increasing the efficiency of which the electrosurgical energy is transferred to the wall “W” of the colon “C”.

With tape 110 prepped, tape 110 is inserted into the colon “C” and navigated to the area of interest, e.g., the diseased tissue to be treated. Tape 110 may be inserted through the anus and navigated through the colon “C” using any suitable surgical tool capable of being inserted into, and navigated through, the colon “C,” such as an endoscope, forceps, or the like. For example, tape 110 may be inserted through a working channel of a suitable endoscope. Once tape 110 has been navigated to the area of interest, tape 110 is adhered to the inner wall “W” of the colon “C.” Alternatively, tape 110 may be first positioned within the colon “C” and, thereafter, leads 120a, 120b engaged with the ends 112a, 112b of conductor 112.

Prior to or after insertion of tape 110 and/or engagement of leads 120a, 120b thereto, the opposite ends of leads 120a, 120b are secured to a resistive heater, or in the case of a procedure using monopolar or bipolar electrosurgical energy, a suitable electrosurgical generator. Energy may then be applied to tape 110, which causes tape 110 to be heated and/or conduct energy through adjacent tissue. With respect to resistive heating embodiments, the heat generated by the energy being applied to tape 110 is transferred to the area of interest to treat the diseased tissue, e.g., via burning, charring, ablating, coagulating, and/or desiccating the diseased tissue. By thermally treating the tissue in this manner, the inflammation and ulceration can be reduced or eliminated entirely, thereby reducing the associated pain and discomfort. With respect to monopolar or bipolar embodiments, the conduction of energy through tissue heats the tissue to effect treatment, e.g., via burning, charring, ablating, coagulating, and/or desiccating the diseased tissue. As noted above, the use of tape 110 localizes the heat and/or energy application to the area of interest and prevents the heat and/or energy from penetrating too deeply within the wall “W” of the colon “C,” thereby preventing unwanted damage to surrounding tissue and/or structure. After tissue is sufficiently treated, leads 120a, 120b are disconnected from conductor 112 or tape 110 and withdrawn from the colon “C.” In embodiments where tape is bio-absorbable, tape 110, including conductor 112, remains in the colon “C” and is gradually dissolved. Alternatively, tape 110 may be removed before, after, or together with leads 120a, 120b.

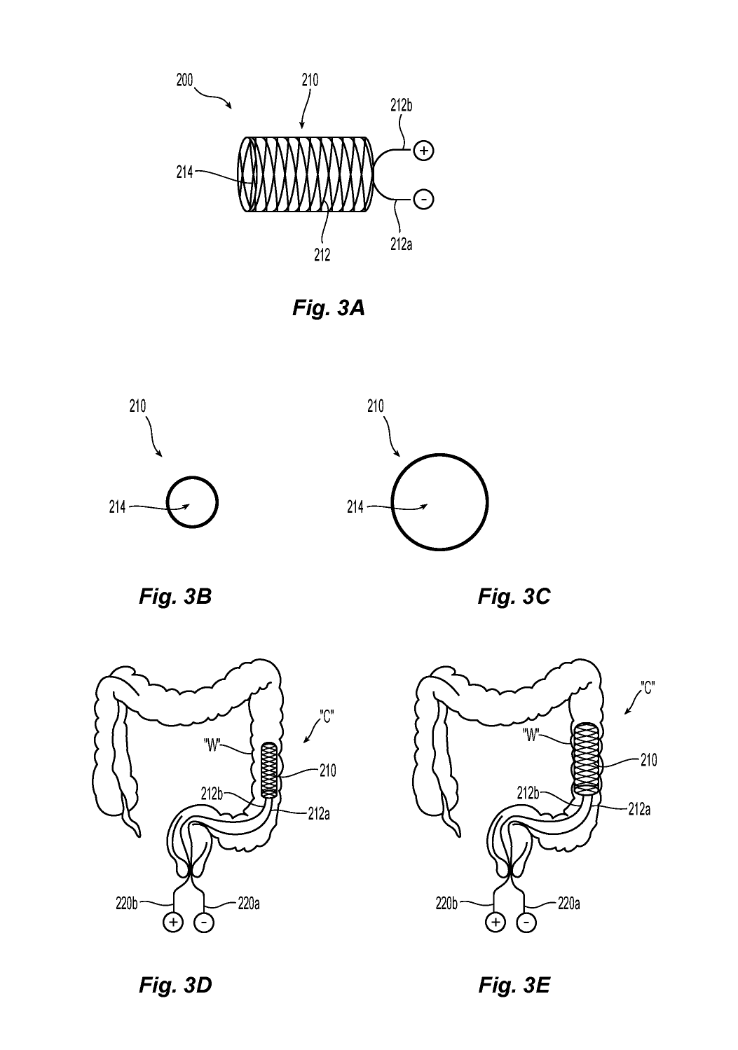

Turning now to FIGS. 3A-3E, another treatment system provided in accordance with the present disclosure and configured to treat diseased bowel tissue is provided generally identified as reference numeral 200. Treatment system 200 is similar to that of treatment system 100 (FIGS. 2A-2B) described above, and therefore, in the interest of brevity only the differences therebetween will be discussed below. Treatment system 200 includes a stent 210 having a conductor 212. Conductor 212 includes first and second ends 212a, 212b configured to connect to first and second leads 220a, 220b. Stent 210 includes a generally cylindrical configuration and may include any suitable diameter or length depending on the size of the area of interest or the procedure being performed. Stent 210 is constructed of any suitable material and, in some embodiments, is formed from a bio-absorbable material. Stent 210 is resilient and is configured to compress or expand due to external stimuli. In this manner, in an initial uncompressed state (FIG. 3C), stent 210 has a first diameter. In a second state (FIG. 3B), where an external stimuli or force is applied to stent 210, the diameter of stent 210 is decreased such that stent 210 may be more easily inserted through the anus and navigated through the colon “C,” as will be discussed in further detail below. As can be appreciated, stent 210 may be compressed to any suitable diameter depending on the size of the orifice stent 210 is being inserted through or the particular procedure being performed. A lumen 214 is defined through stent 210 having a diameter that is configured for the passage of waste therethrough while stent 210 is left in the colon “C” to treat tissue and, ultimately dissolve or be retrieved at a later time.

Stent 210 may be constructed from polymers, suitable resilient metals, composites, or the like and may be monolithically formed or formed from a mesh type construction. In this manner, it is contemplated that stent 210 may be formed from a homogeneous construction or a heterogeneous construction (i.e., a combination of metallic and non-metallic bio-absorbable materials). Conductor 212 may include any or all of the features of any of the embodiments of conductor 112 (FIGS. 2A-2B) and, therefore, in the interest of brevity, conductor 212, and the operation thereof, will not be discussed hereinbelow.

In use, treatment system 200 may be utilized in a similar respect to treatment system 100 (FIG. 2C) as detailed above, with the exception that stent 210 is initially compressed by an external stimuli before advancing stent 210 within the colon (FIG. 3D). Once stent 210 has been maneuvered to the area of interest, the external stimuli is removed and stent 210 is permitted to expand against the inner wall “W” of the colon “C” and against the area of interest (FIG. 3E). Thereafter, energy is applied to treat tissue, similarly as detailed above.

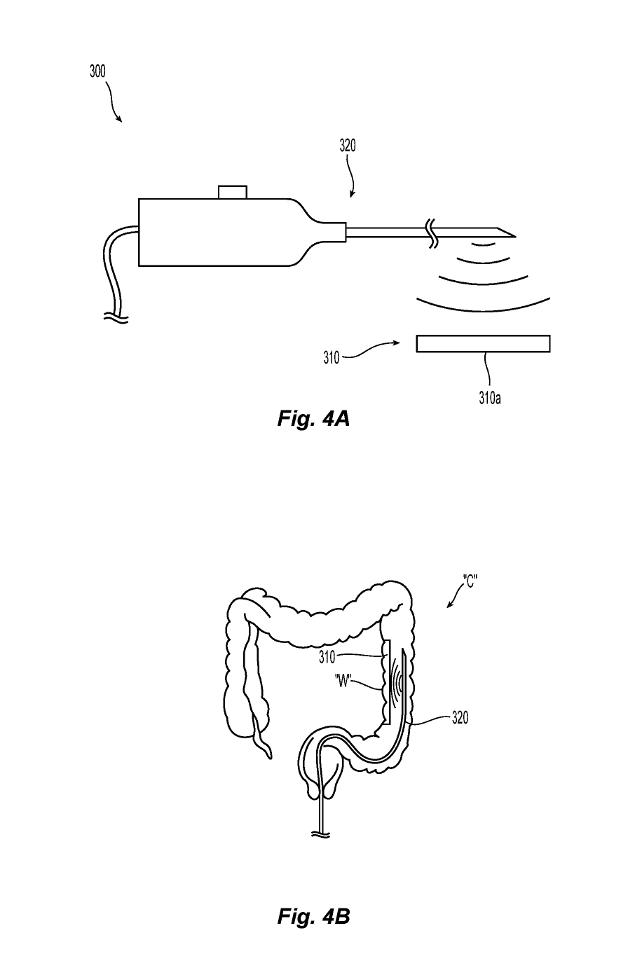

Referring to FIGS. 4A and 4B, another treatment system provided in accordance with the present disclosure and configured for use in treating the bowel is shown generally identified as reference numeral 300. Treatment system 300 generally includes a treatment pad 310 and an antenna 320 capable of being advanced within the colon “C” and configured to emit energy, e.g., radio frequency energy, microwave energy, etc.

As illustrated in FIG. 4A, although shown as generally having a rectangular cross section, it is contemplated that treatment pad 310 may include any suitable cross section, such as convex, concave, or the like. Treatment pad 310 is constructed of any suitable material, e.g., a resilient bio-absorbable material, capable of absorbing energy from antenna 320 or acting as a screen to limit the depth of energy transmission from antenna 320 through treatment pad 310. Treatment pad 310 may define any suitable configuration such as rectangular, square, circular, oval, or the like. Treatment pad 310 may be monolithically formed or formed from a screen type configuration (i.e., weave or cross hatch configuration), depending on the type of tissue being treated or the needs of the procedure being performed. Treatment pad 310 may be formed from a homogeneous construction or a heterogeneous construction (i.e., a combination of metallic and non-metallic bio-absorbable materials). A bottom surface 310a of treatment pad 310 is configured to be selectively secured to an inner wall “W” of the colon “C,” for example, using bio-absorbable adhesives or mechanical elements such as hooks or the like. As noted above, treatment pad 310 may be configured to absorb energy emitted by the antenna 320 and become heated, thereby treating tissue in contact therewith. Alternatively or additionally, treatment pad 310 is configured to act as a screen. In this manner, energy emitted by the antenna 320 and passing through treatment pad 310 is concentrated to the area of interest and is prevented from penetrating too deeply within the wall “W” of the colon “C,” thereby preventing unwanted damage to surrounding tissue and/or structure.

Antenna 320 may be any suitable antenna and may be configured to emit any suitable energy e.g., radio frequency energy, microwave energy, etc., for treating tissue of the colon “C.” As one example, antenna 320 may include a side firing antenna, such that energy is only directed in a direction perpendicular to an longitudinal axis defined by antenna 320. Antenna 320 is configured to communicate with a suitable generator (not shown) for supplying energy thereto.

As illustrated in FIG. 4B, in use, once an area of interest is identified and the colon “C” has been prepared, treatment pad 310 is inserted into the colon “C” and navigated to the area of interest. Treatment pad 310 may be manipulated in any suitable manner in order to advance treatment pad 310 into the colon “C” (e.g., folding, compressing, or the like). Treatment pad 310 may be inserted through the anus and navigated through the colon “C” to the area of interest using any suitable surgical tool capable of being inserted into and navigated through the colon “C” such as an endoscope, forceps, or the like. For example, treatment pad 310 may be inserted through a working channel of a suitable endoscope.

Once treatment pad 310 has been navigated to the area of interest, treatment pad 310 is adhered to the inner wall “W” of the colon “C.” At this point, antenna 320 is advanced within the colon “C” and navigated to the area of interest. Next, antenna 320 is activated to emit energy therefrom. The energy emitted from antenna 320 is absorbed or screened by treatment pad 310. In embodiments where the energy is absorbed, the heat generated by the energy being applied to treatment pad 310 is transferred to the area of interest to treat the diseased tissue, e.g., via burning, charring, ablating, coagulating, and/or desiccating the diseased tissue. In embodiments where treatment pad 310 functions as a screen, energy is transmitted through treatment pad 310 through tissue to a relatively shallow depth, to effect treatment, e.g., via burning, charring, ablating, coagulating, and/or desiccating the diseased tissue, without damaging underlying tissue. By treating the tissue in either of these manners, the inflammation and ulceration can be reduced or eliminated entirely, thereby reducing the associated pain and discomfort. Selective control of the frequency and power settings of the energy may be utilized to ensure sufficient heating of tissue while inhibiting damage to surrounding tissue and critical structures. In this manner, the use of treatment pad 310 localizes the energy application to the area of interest an prevents the energy from penetrating too deeply within the wall “W” of the colon “C,” thereby preventing unwanted damage to surrounding tissue and/or structure.

After the application of energy is complete, antenna 320 is removed from the colon “C.” Treatment pad 310 is left adhered to the inner wall “W” of the colon “C” to be gradually dissolved. In embodiments where treatment pad 310 is not bio-absorbable, it is contemplated that treatment pad 310 be removed from the area of interest at the completion of the procedure.

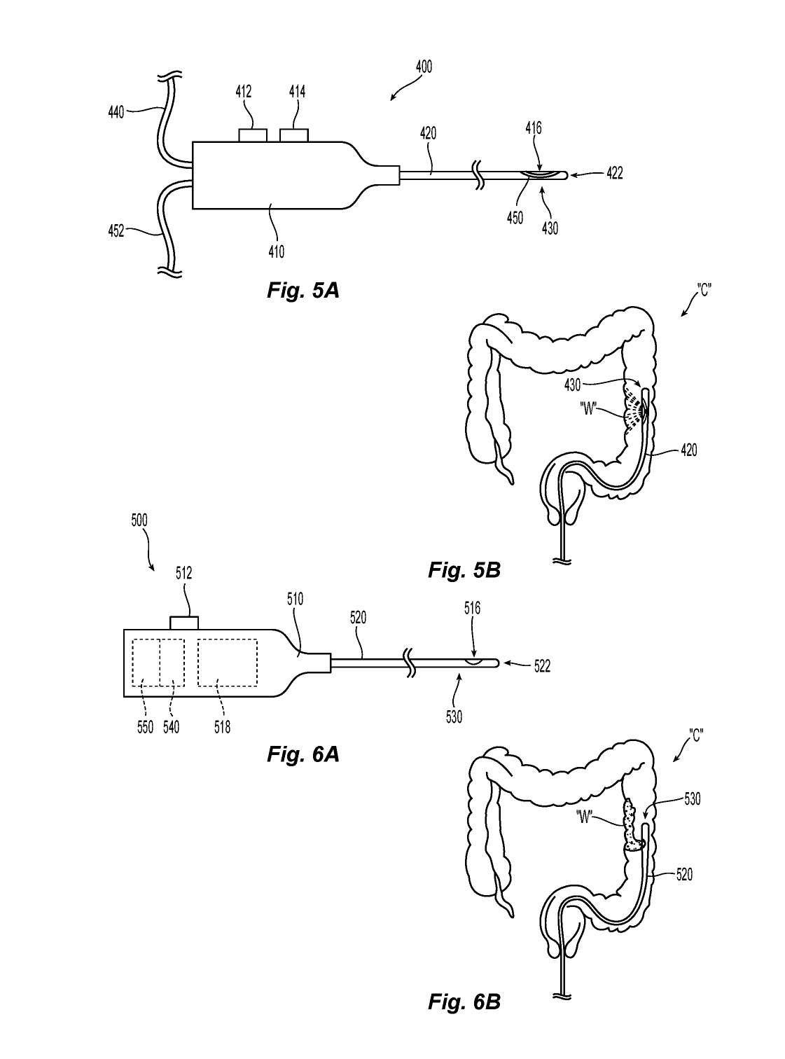

FIGS. 5A and 5B illustrate an instrument 400 provided in accordance with the present disclosure and configured for use in treating the bowel. Instrument 400 generally includes a handle 410, a shaft 420 extending distally from handle 410, and an end effector 430 supported on a distal end of shaft 420. Shaft 420 may be constructed from a suitable resilient material such that shaft 420 may facilitate positioning within the colon “C” (FIG. 5B). Shaft 420 terminates in a blunt distal end 422 that may include any suitable profile capable of inhibiting tissue penetration, such as planar. Shaft 420 is configured to be advanced and navigated within the colon “C.” More specifically, shaft 420 may be configured to be inserted within a working channel of a suitable surgical instrument, such as an endoscope.

Instrument 400 further includes a fluid tube 440 configured to selectively couple a fluid source or reservoir (not shown) containing a fluid or paint capable of absorbing and/or being excited by energy and being heated in response thereto e.g., a paint containing nano-particles suspended therein. In this manner, the fluid absorbs and/or is excited by energy, is heated and, in turn, heats the portion of the inner wall “W” of the colon “C” in contact with the fluid, thereby localizing the energy application to the area of interest and preventing the energy from penetrating too deeply within the wall “W” of the colon “C.”

Fluid tube 440 extends into handle 410 and through shaft 420. Fluid tube 440 terminates at an opening or nozzle 416 defined through an outer surface of end effector 430. Nozzle 416 is in fluid communication with fluid tube 440 and is configured to spray or eject fluid on an area of interest located on the inner wall “W” of the colon “C.” Nozzle 416 may atomize the fluid such that inner wall “W” of the colon “C” is evenly coated with the fluid. As an alternatively to having fluid tube 440 extend proximally from handle 410, the fluid source or reservoir (not shown) can be disposed within handle 410. In embodiments, the shaft 420 and/or nozzle 416 may be rotatable with respect to handle 410 to enable a clinician to more easily treat various areas of interest.

As illustrated in FIG. 5A, end effector 430 further includes a radiating portion 450. Radiating portion 450 is concentric with nozzle 416, such that nozzle 416 penetrates a center region of radiating portion 450. Radiating portion 450 is in electrical communication with a suitable generator capable of generating energy, e.g., monopolar or bipolar radiofrequency energy, microwave energy, etc., via electrical cord 452. The generator may alternatively be disposed within handle 410 of instrument 400. A first switch 412, or other suitable activation member, is disposed on handle 410 and is operably coupled to the generator. Switch 412 permits selective actuation of the generator, thereby providing control over the application of energy being applied by radiating portion 450.

A second switch 414, or other suitable activation member, is disposed adjacent to first switch 412 on handle 410 and is operably coupled to a pump (not shown). The pump is in fluid communication with the reservoir such that when second switch 414 is activated, the pump pressurizes the fluid and expels it through nozzle 416. For example, the pump may continuously pressurize the fluid while second switch 414 may be a suitable valve or solenoid capable of selectively interrupting flow through fluid tube 440.

Referring to FIG. 5B, in use, a fluid or paint is loaded into the reservoir (not shown). At this point, fluid tube 440 may be primed with fluid (i.e., the fluid is pressurized by a pump (not shown) and advanced within fluid tube 440 and through nozzle 416 such that any air contained therein is expelled). Once an area of interest is identified and the colon “C” prepared, shaft 420, led by end effector 430, is advanced into the colon “C” and navigated to the area of interest. Shaft 420 may be inserted through the anus and navigated through the colon “C” to the area of interest using any suitable surgical tool, such as those detailed above.

Once shaft 420 has been navigated to the area of interest, second switch 414 is activated, thereby expelling the fluid out of nozzle 416 and coating the inner wall “W” of the colon “C” having the diseased tissue. At this point, the fluid is absorbed by the diseased tissue of the colon “C.” Next, first switch 412 is activated to emit energy from radiating portion 450. The energy is absorbed by and/or excites the fluid, causing the fluid to increase in temperature to the point where the heated fluid is capable of treating the diseased tissue, e.g., via burning, charring, ablating, coagulating, and/or desiccating the diseased tissue. By thermally treating the tissue in this manner, the inflammation and ulceration can be reduced or eliminated entirely, thereby reducing the associated pain and discomfort. The particular fluid provided, e.g., the composition and/or concentration of the nano-particles disposed therein, and selective control of the frequency and power settings of the energy applied may be utilized to ensure sufficient heating of the tissue to be treated while inhibiting damage to surrounding tissue and critical structures. Additionally, the use of the fluid diffuses the energy evenly across the area of interest and localizes the energy application to the area of interest. In this manner, the energy is prevented from penetrating too deeply within the wall “W” of the colon “C”, thereby preventing unwanted damage to surrounding tissue and/or structure. After the application of energy is complete, shaft 420 is removed from the colon “C”. The fluid may thereafter be absorbed by the wall “W” of the colon “C,” or flushed out of the patient.

Instrument 400 may subsequently be used to treat other diseased areas of the colon “C” in either an incremental fashion or a continuous fashion. More specifically, with respect to use in an incremental fashion, after treatment as detailed above, shaft 420 may be further advanced within the colon “C” or rotated at the previous location. Thereafter, second switch 414 may be activated to coat the inner wall “W” of the colon “C” at the new area of interest. The procedure discussed above may then be followed. Alternatively, with respect to use in a continuous fashion, shaft 420 may be advanced within the colon “C” or rotated at the area of interest while simultaneously activating second switch 414. In this manner, a continuous length or circumference of the inner wall “W” of the colon “C” is coated with fluid. Thereafter, shaft 420 may re-trace the path taken previously during the coating procedure to apply electrosurgical energy. It is contemplated that a first instrument (not shown) capable of expelling fluid and a second, separate instrument (not shown) capable of emitting energy may be utilized, instead of instrument 400.

FIGS. 6A and 6B illustrate another instrument 500 provided in accordance with the present disclosure and configured for use in treating the bowel. Instrument 500 generally includes a handle 510, a shaft 520 extending distally from the handle 510, and an end effector 530 disposed on a distal end of shaft 520. Shaft 520 may be constructed similarly as shaft 420 of instrument 400 (FIGS. 5A-5B) and may be configured for use with an insertion instrument, such as those detailed above.

Instrument 500 further includes a fluid source or reservoir 518 configured to store a fluid, gel, wax, or other suitable viscous material, therein. A fluid tube (not shown) extends from a first end, in communication with the reservoir 518, through instrument 500, to a second end that terminates in an opening or nozzle 516 defined through an outer surface of end effector 530. Reservoir 518 is configured to heat the fluid stored therein, e.g., using a resistive heater or the like. Alternatively or additionally, the fluid may be heated while being pumped through the fluid tube by passing through an inline heater (not shown) such as a cartridge heater or the like. The fluid is heated to a suitable temperature that is capable of treating diseased tissue by burning, charring, coagulating, and/or desiccating the diseased tissue. Nozzle 516 is configured to eject the fluid onto the inner wall “W” or the colon “C” in order to treat the diseased tissue at the area of interest. The fluid is pressurized and ejected through nozzle 516 using a suitable pump or motor 540 disposed within handle 510. The pump 540 is in fluid communication with reservoir 518 and a battery or other suitable electrical source 550. Battery 550 is also disposed within handle 510 and is in electrical communication with the heater and reservoir 518, thereby enabling instrument 500 to be wireless. However, it is also contemplated that reservoir 518, battery 550, motor 540, and/or any combination thereof, be disposed remote from the instrument 500.

A switch 512, or other suitable activation member, is disposed on handle 510 and is operably coupled to the pump 540. Switch 512 permits selective dispensing of fluid through nozzle 516 and onto an area of interest.

With reference to FIG. 6B, in use, a fluid is initially loaded into the reservoir 518. At this point, the fluid is heated by the heater (not shown) to a temperature that is suitable for treating diseased tissue. Next, the instrument is primed with fluid (i.e., the fluid is pressurized and advanced within the fluid tube (not shown) and through nozzle 516 such that any air contained therein is expelled). Once an area of interest is identified and the colon “C” prepared, shaft 520, led by end effector 530, is advanced into the colon “C” and navigated to the area of interest, e.g., similarly as detailed above.

Once shaft 520 has been navigated to the area of interest, switch 512 is activated, thereby expelling the fluid out of nozzle 516 and coating the inner wall “W” of the colon “C” having the diseased tissue. At this point, the diseased tissue is heated by the fluid and is treated via burning, charring, coagulating, and/or desiccating. By thermally treating the tissue in this manner, the inflammation and ulceration can be reduced or eliminated entirely, thereby reducing the associated pain and discomfort. The particular fluid provided and selective control of the heating of the fluid may be utilized to ensure sufficient heating of the fluid while inhibiting damage to surrounding tissue and critical structures. Additionally, the use of the fluid causes even distribution of the energy to the diseased tissue and localizes the energy application to the area of interest. In this manner, the energy is prevented from penetrating too deeply within the wall “W” of the colon “C” and prevents unwanted damage to surrounding tissue and/or structure.

Use of a viscous fluid such as a gel, or a fluid that is converted to a solid or semi-solid upon cooling, such as a wax, is particularly beneficial in that the area of interest remains coated by the fluid (or solidified fluid) for a sufficient period of time after treatment in order to protect the treated area of the colon “C” during healing. Thereafter, the fluid adhered to the inner wall “W” of the colon “C” sloughs off and/or is removed and subsequently expelled from the colon “C.”

Instrument 500 may subsequently be used to treat other diseased areas of the colon “C” in either an incremental fashion or a continuous fashion. More specifically, with respect to use in an incremental fashion, after treatment as detailed above, shaft 520 may be further advanced within the colon “C” or rotated at the previous location. Thereafter, switch 512 may be activated to coat the inner wall “W” or the colon “C” at the new area of interest. Alternatively, with respect to use in a continuous fashion, shaft 520 may be advanced within the colon “C” or rotated at the area of interest while simultaneously activating switch 512. In this manner, a continuous length or circumference of the inner wall “W” of the colon “C” is coated with fluid.

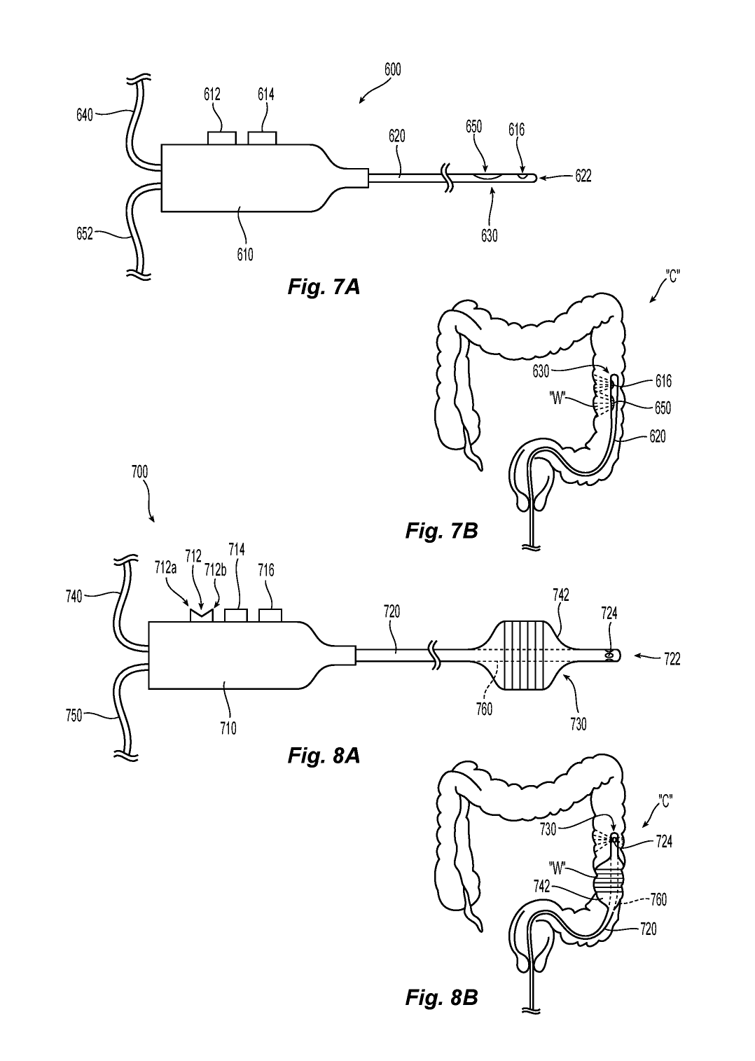

FIGS. 7A and 7B illustrate still another instrument 600 provided in accordance with the present disclosure and configured for use in treating the bowel. Instrument 600 generally including a handle 610, a shaft 620 extending distally from handle 610, and an end effector 630 supported on a distal end of shaft 620. Shaft 620 may similar to the shafts of the instruments detailed above.

Instrument 600 further includes a fluid tube 640 configured to selectively engage a fluid source or reservoir (not shown) storing a bio-absorbable fluid capable of absorbing or reacting to exposure to light, such as a photosensitizer or a fluid containing nano-particles suspended therein. Fluid tube 640 extends from the reservoir into handle 610 and through shaft 620. Fluid tube 640 terminates at an opening or nozzle 616 defined through an outer surface of end effector 630. Nozzle 616 is in fluid communication with fluid tube 640 and is configured to spray or eject fluid on an area of interest located on the inner wall “W” of the colon “C.” Nozzle 616 may atomize the fluid such that the inner wall “W” of the colon “C” is evenly coated with the fluid. As an alternative to connecting to the reservoir with fluid tube 640, the reservoir may be disposed within handle 610.

As illustrated in FIG. 7A, end effector 630 further includes a radiating portion 650. Radiating portion 650 is disposed on end effector 630 at a location proximal to nozzle 616. Radiating portion 650 is coupled to a suitable light source generator (not shown) capable of generating a light source having a wavelength that is readily absorbed by the fluid (i.e., laser light, light emitting diode, etc.) via a fiber optic cable or other suitable light carrying device 652. Alternatively, a light emitting diode may be disposed within radiating portion 650, thereby eliminating the need for a separate light source generator located remotely from the instrument 600.

A first switch 612, or other suitable activation member, is disposed on handle 610 and is operably coupled to the light source generator. Switch 612 permits selective actuation of the light source generator, thereby providing control over the application of light being emitted by radiating portion 650.

A second switch 614, or other suitable activation member, is disposed adjacent to first switch 612 on handle 610 and is operably coupled to a pump (not shown). The pump is in fluid communication with the reservoir and fluid tube 640 such that when second switch 614 is activated, the pump pressurizes the fluid and expels it through nozzle 616. The pump and second switch 615 may be similar to those detailed above.

Referring to FIG. 7B, in use, a fluid is initially loaded into the reservoir and fluid tube 640 primed with fluid. Once an area of interest is identified and the colon “C” prepared, shaft 620, led by end effector 630, is advanced into the colon “C” and navigated to the area of interest, e.g., as detailed above.

Once shaft 620 has been navigated to the area of interest, second switch 614 is activated, thereby expelling the fluid out of nozzle 616 and coating the inner wall “W” of the colon “C” having the diseased tissue. Next, first switch 612 is activated to emit light from radiating portion 650 having a wavelength that is readily absorbed by the fluid. The light absorbed by the fluid causes the fluid to excite, in the case of a photosensitizer, or heat up, in the case of a fluid containing nano-particles, thereby treating the diseased tissue by burning, charring, coagulating, and/or desiccating the tissue with the heated fluid. By thermally treating the tissue in this manner, the inflammation and ulceration can be reduced or eliminated entirely, thereby reducing the associated pain and discomfort. The use of the fluid causes even distribution of the energy to the diseased tissue and localizes the energy application to the area of interest. In this manner, the energy is prevented from penetrating too deeply within the wall “W” of the colon “C” and prevents unwanted damage to surrounding tissue and/or structure. After the application of light is complete, shaft 620 is removed from the colon “C.” The fluid is eventually dissolved or may be flushed out. Instrument 600 may subsequently be used to treat other diseased areas of the colon “C” in either an incremental fashion or a continuous fashion, similarly as detailed above.

It is further contemplated that the light emitted from radiating portion 650 may be used to heat an area of interest to treat the diseased tissue. In this manner, no fluid is dispensed from nozzle 616. Rather, end effector 630 may be navigated through the colon “C” to the area of interest and thereafter, first switch 612 may be activated to expose the area of interest with light. The intensity of the light may be sufficient to treat the diseased tissue by burning, charring, ablating, coagulating, and/or desiccating the diseased tissue.

Turning now to FIGS. 8A and 8B, still another instrument 700 is provided in accordance with the present disclosure and configured for use in treating the bowel. Instrument 700 generally includes a handle 710, a shaft 720 extending distally from handle 710, and an end effector 730 disposed on a distal end thereof. Shaft 720 may be constructed similarly as detailed above with respect to the previous embodiments.

Instrument 700 further includes a first fluid tube 740 configured to selectively engage a fluid source or reservoir (not shown) storing a fluid capable of absorbing or reacting to exposure to light, such as a photosensitizer or a fluid containing nano-particles suspended therein. First fluid tube 740 extends into handle 710 and through shaft 720 and is fluidly coupled to a plurality of ports 724 defined through a distal end of end effector 730. Ports 724 are disposed at various radial and/or longitudinal positions along the distal end of end effector 730 and are configured to spray or eject fluid on an area of interest located on the inner wall “W” of the colon “C.” Ports 724 may atomize the fluid such that the inner wall “W” of the colon “C” is evenly coated with the fluid. As an alternative to an external reservoir, the reservoir may be disposed within handle 710.

As illustrated in FIG. 8A, end effector 730 further includes a balloon 742 disposed at a proximal location in relation to the plurality of ports 724. Balloon 742 is formed from a resilient material and is configured to diffuse light, such that any light that comes in contact with balloon 742 is evenly distributed throughout balloon 742. Balloon is coupled to a second fluid tube 750 that is configured to connect to an inflation source (not shown). Second fluid tube 750 extends into handle 710 and through shaft 720 and couples balloon 742 to the inflation source such that balloon 742 is in fluid communication therewith. In this manner, an inflation fluid, e.g., air, may be delivered to and removed from balloon 742 to cause balloon 742 to inflate or deflate. A rocker switch 712, or other suitable activation member, is disposed on handle 710 and is operably coupled to the inflation source and/or second fluid tube 750 to enable selective inflation and deflation of balloon 742. In this manner, when a first portion 712a of rocker switch 712 is activated, pressure is supplied to pump fluid through second fluid tube 750 and into balloon 742, thereby inflating balloon 742. Alternatively, when a second portion 712b of rocker switch 712 is activated, suction is applied to withdraw fluid from balloon 742, thereby deflating balloon 742.

Continuing with reference to FIG. 8A, a radiating portion 760 is disposed on end effector 730 internal to balloon 742 such that balloon 742 entirely encompasses radiating portion 760. Radiating portion 760 is configured to emit light in a radial direction towards balloon 742, such that the entire circumference of radiating portion 760 emits light. Radiating portion 760 is coupled to a suitable light source generator (not shown) capable of generating a light source having a wavelength that is readily absorbed by the fluid (e.g., laser light, light emitting diode, etc.) via a fiber optic cable or other suitable light carrying device (not shown). Alternatively, a light emitting diode may be disposed within radiating portion 760, thereby eliminating the need for a separate light source generator located remotely from the instrument 700. The light source generator may be disposed within handle 710 and coupled to radiating portion 760 by a fiber optic cable or other suitable light carrying device.

A first switch 714, or other suitable activation member, is disposed on handle 710 adjacent to rocker switch 712 and is operably coupled to the light source generator. First switch 714 permits selective actuation of the light source generator, thereby providing control over the application of light being emitted by radiating portion 760.

A second switch 716 is disposed adjacent to first switch 714 on handle 710 and is operably coupled to a pump (not shown). The pump, which may be similar to the pumps detailed above, is in fluid communication with the reservoir such that when second switch 716 is activated, the pump pressurizes the fluid and expels it through the plurality of ports 724.

Referring now to FIG. 7B, in use, a fluid is initially loaded into the reservoir and fluid tube 640 primed with fluid. Once an area of interest is identified and the colon “C” prepared, shaft 720, led by end effector 730, is advanced into the colon “C” and navigated to the area of interest, e.g., as detailed above.

Once shaft 720 has been navigated to the area of interest, second switch 716 is activated, thereby expelling the fluid out of the plurality of ports 724 and coating the inner wall “W” of the colon “C” having the diseased tissue. Next, shaft 720 is further advanced until balloon 742 is located adjacent to the coated inner wall “W” of the colon “C” (i.e., the balloon is centered within the coated portion of the inner wall “W” of the colon “C”). At this point, the first portion 712a of the rocker switch 712 is activated to supply inflation fluid from the inflation source (not shown) through second fluid tube 750 to balloon 742, thereby inflating balloon 742. Balloon 742 is sufficiently inflated so as to engage the inner wall “W” of the colon “C” that has been coated with fluid.

Thereafter, first switch 714 is activated to emit light having a wavelength that is readily absorbed by the fluid from radiating portion 760. The light absorbed by the fluid causes the fluid to excite, in the case of a photosensitizer, or heat up, in the case of a fluid containing nano-particles, thereby treating the diseased tissue of the colon “C.” By thermally treating the tissue in this manner, the inflammation and ulceration can be reduced or eliminated entirely, thereby reducing the associated pain and discomfort. Further, the treatment of tissue is localized to the area coated by the fluid, thereby preventing damage to surrounding tissue and/or structure.

After the application of light is complete, the second portion 712b of rocker switch 712 is activated to withdraw the fluid from balloon 742 and therefore deflate balloon 742. At this point, shaft 720 is removed from the colon “C.” and the fluid left to dissolve or expelled.

Instead of removing shaft 720, instrument 700 may subsequently be used to treat other diseased areas of the colon “C” in an incremental fashion. More specifically, after treatment as detailed above, shaft 720 may be further advanced within the colon “C.” Thereafter, the procedure detailed above may be repeated. It is contemplated that the above described procedure may be repeated as many times as necessary to treat a single or plurality of areas of interest. Continuous treatment is also contemplated, wherein second switch 716 is continuously or periodically activated to expel the fluid out of the plurality of ports 724, first switch 714 is continuously or periodically activated to emit light, and shaft 720 is moved, led by the free end thereof, through the colon “C” such that, as shaft 720 is moved, tissue is coated and subsequently treated. In such embodiments, the balloon 742 may be inflated sufficiently so as to engage the inner wall “W” of the colon “C” while also allowing balloon 742 to be urged through the colon “C.”

The above described procedure may also be performed without initially coating the inner wall “W” of the colon “C” with fluid. In this manner, once shaft 720 has been navigated to the area of interest, the balloon 742 may be inflated so as to engage the inner wall “W” of the colon “C,” as discussed above. At this point, first switch 714 may be activated to emit light from radiating portion 760. The light emitted from radiating portion 760 is diffused by balloon 742, thereby evenly distributing the energy from the light across the portion of balloon 742 in contact with the inner wall “W” of the colon “C.” The energy absorbed by balloon 742 is then transferred to the inner wall “W” of the colon “C,” thereby causing the inner wall “W” to heat up and treat the diseased tissue, e.g., via burning, charring, ablating, coagulating, and/or desiccating the diseased tissue. Any combination of the above noted procedures may be performed. Specifically, the procedure utilizing a fluid may be performed at a first area of interest, and the second procedure utilizing only light may be utilized at a second area of interest, and vice versa.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An apparatus for treating inflammatory bowel diseases, comprising:
a handle;
a shaft extending distally from the handle, the shaft configured to be advanced within a patient's colon; and
an end effector disposed at a distal end of the shaft, the end effector including a radiating portion configured to emit energy and a nozzle configured to emit a fluid.

2. The apparatus according to claim 1, wherein the radiating portion and the nozzle are disposed in a concentric orientation with the nozzle disposed within the radiating portion.

3. The apparatus according to claim 1, wherein the nozzle is disposed distal of the radiating portion.

4. The apparatus according to claim 1, wherein the end effector includes an expanding portion, the expanding portion configured to expand to engage an inner wall of a patient's colon.

5. The apparatus according to claim 4, wherein the radiating portion is disposed within the expanding portion.

6. The apparatus according to claim 5, wherein the expanding portion is configured to diffuse light.

7. The apparatus according to claim 1, wherein the handle includes a first switch disposed thereon operably coupled to the radiating portion, wherein activating the first switch causes the radiating portion to emit energy therefrom.

8. The apparatus according to claim 7, wherein the handle includes a second switch disposed thereon operably coupled to the nozzle, wherein activating the second switch causes the nozzle to emit fluid therefrom.

9. The apparatus according to claim 1, wherein the radiating portion is configured to emit radio frequency, microwave, or light energy.

10. The apparatus according to claim 1, wherein the fluid is a fluid containing nano-particles or a photosensitizer.

11. An apparatus for treating inflammatory bowel diseases, comprising:
a body configured to be advanced within a patient's colon;
a conductor disposed on the body, the conductor capable of transmitting energy therethrough; and
at least one terminal disposed on an end of the conductor, the terminal configured to selectively engage an energy source.

12. The apparatus according to claim 11, wherein the body defines a generally flat configuration including an adhesive disposed on a surface thereof, the adhesive configured to adhere the body to an inner wall of a patient's colon.

13. The apparatus according to claim 11, wherein the body defines a cylindrical configuration, wherein the body is biased towards an expanded configuration for engaging an inner wall of a patient's colon, and is configured to be compressed by an external stimuli.

14. The apparatus according to claim 11, wherein the conductor is configured to be resistively heated via the energy source to heat tissue adjacent thereto or to conduct electrical energy from the energy source through tissue adjacent thereto.

15. A method of treating inflammatory bowel diseases, comprising:
coating an inner wall of a patient's colon with a fluid; and
emitting energy towards the fluid to heat the fluid, thereby thermally treating tissue of a patient's colon in contact with the fluid.

16. The method according to claim 15, wherein emitting energy towards the fluid includes expanding an expandable portion of an end effector of a surgical instrument such that the expanding portion engages an inner wall of a patient's colon.

17. The method according to claim 16, wherein emitting energy includes diffusing light energy through the expanding portion to heat tissue in contact with the expanding portion.

18. The method according to claim 15, wherein coating an inner wall of a patient's colon includes ejecting the fluid from a nozzle disposed on a distal end of an end effector of a surgical instrument.

19. The method according to claim 18, wherein the fluid includes nano-particles suspended therein to facilitate absorption of the energy, and wherein emitting the energy towards the fluid includes exciting the nano-particles to heat up the fluid.

20. The method according to claim 18, wherein the fluid is a photo sensitizer, and wherein emitting the energy towards the fluid includes emitting light energy to excite the photosensitizer fluid.
